(19) United States
(12) Patent Application Publication
(10) Pub. No.: US 2024/0058229 A1
(43) Pub. Date: Feb. 22, 2024

(54) PREPARATION OF A TINTED COSMETIC COMPOSITION

(71) Applicant: COATEX, Genay (FR)

(72) Inventors: Sandie BOURSET, Montluel (FR); Anne ESCOFFIER, Fontaines Saint Martin (FR); Helene PERNOT, Lyon (FR)

(73) Assignee: COATEX, Genay (FR)

(21) Appl. No.: 18/260,963

(22) PCT Filed: Mar. 4, 2022
(86) PCT No.: PCT/FR2022/000015

§ 371 Date: Jul. 11, 2023

(30) Foreign Application Priority Data
Mar. 5, 2021 (FR) FR2102146

(51) Int. Cl. A61K 8/06; A61K 8/81; A61Q 1/02

(52) U.S. Cl. CPC A61K 8/062; A61K 8/8152; A61Q 1/02; A61K 2800/434; A61K 2800/5424; A61K 2800/95

(57) ABSTRACT

A method may prepare a tinted cosmetic composition, in particular a make-up composition, which is particularly versatile. Such a method may make it possible to prepare tinted cosmetic compositions that can be suitable for many types of skin tone, e.g., including preparing a continuous hydrophilic phase comprising (i) a hydrophilic compound A in a range of from 90 to 99.6 wt. % of a hydrophilic phase of the oil-in-water emulsion, and (ii) a polymer P, in a range of from 0.4 to 10 wt. % of the hydrophilic phase, the polymer P comprising, in polymerized form, an anionic monomer (a1) and a C₁-C₈ ester (a2); adding, under stirring, a lipophilic phase comprising a lipophilic compound B, to form a dispersion of lipophilic phase particles in the continuous hydrophilic phase; then adding, under stirring, of a pigment C. The coloration, retention, and stability properties of such compositions are particularly improved.

The invention relates to a method for preparing a tinted cosmetic composition, in particular a make-up composition, which is particularly versatile. The method of the invention makes it possible to prepare tinted cosmetic compositions that can be suitable for many types of skin tones. The colouration, retention and stability properties of these compositions are particularly improved.

In the field of cosmetic compositions, the compositions formed from a lipophilic emulsion phase in a hydrophilic phase are particularly appreciated, in particular for the preparation of tinted cosmetic compositions. These tinted cosmetic compositions are generally creams.

These creams are intended for many uses, in particular as foundation, for correcting the complexion or for unifying the complexion. These uses can be combined with a care effect or a hydration effect.

While there is a trend towards simplifying the lists of ingredients used, cosmetic compositions in the form of emulsions generally comprise numerous ingredients that are added to the hydrophilic and lipophilic compounds that make up the base of an emulsion.

The absence of certain ingredients commonly used in cosmetic compositions is therefore sought. In particular, the absence of surface-active compounds in a cosmetic composition can be an advantage when preparing it or when using it. However, the elimination of such surface-active compounds, particularly surface-active compounds that are solid at room temperature, can lead to major or prohibitive difficulties in the preparation, storage or use of cosmetic compositions.

Among the lipophilic ingredients used in tinted cosmetic compositions, it can be advantageous to limit or eliminate silicone oils. Eliminating or limiting the use of organic solvents in cosmetic compositions is also a highly desirable property.

The compatibility of the various ingredients in cosmetic compositions remains an important property for these compositions. In particular, the combined use of a variety of pigments must be sought, whether these pigments are hydrophilic or lipophilic, whether they are of natural or synthetic origin, whether they are surface treated or not. Improved compatibility must make it possible to improve the resistance to dephasing of tinted cosmetic compositions. Homogeneity of the tinted cosmetic compositions is also sought in order to avoid the presence of pigment aggregates that can cause coloured streaks when applied.

Tinted cosmetic compositions should be capable of comprising a high concentration of pigments. The ability to increase the pigment concentration of tinted cosmetic compositions is particularly sought in order to provide compositions that can be used on skins with a higher or lower level of skin tone.

Tinted cosmetic compositions are most often used on the skin. They must have a variety of properties during their preparation as well as during their storage or their transport and even when they are used, in particular during and after this application. When applied to the skin, cosmetic compositions should leave a pleasant, fresh feeling and avoid a greasy or oily effect, particularly a sticky, greasy effect.

The complexity of tinted cosmetic compositions generally requires the use of complex preparation methods. The efficacy and simplification of these preparation methods are major objectives. Eliminating or limiting any heating steps is a significant advantage. The use of conventional equipment, in particular conventional stirring equipment with low or average shear gradients, is also highly advantageous and makes it possible to dispense with the need to use equipment that operates at high or very high shear gradients. The ability to introduce pigments into either the lipophilic or the hydrophilic phase is also a significant advantage. The search for methods for preparing tinted cosmetic compositions therefore aims to find effective, versatile and universal methods.

When used, in particular when applied to the skin, tinted cosmetic compositions should have a high covering capacity while limiting the amount of composition applied. Pigment colour retention on the skin also needs to be improved, in particular according to the type or the tone of the skin to which the composition is applied.

The appearance of the tinted cosmetic compositions when applied is also increasingly important. Generally, these compositions should not leave an oily, opaque or shiny appearance on the skin but they should allow the skin to retain an appearance that is as natural, even, glowing and matte as possible. After application, the stability of the pigments within the tinted cosmetic composition is also very important.

Document WO2019185999 describes the preparation of an oil-in-water emulsion.

The methods for preparing tinted cosmetic compositions of the prior art do not provide satisfactory solutions to these problems. There is thus a need to provide improved methods for obtaining more effective and more efficient tinted cosmetic compositions.

Thus, the invention provides a method for preparing a tinted cosmetic composition in the form of an oil-in-water emulsion prepared in the absence of surface-active compounds, comprising:
the preparation of a continuous hydrophilic phase comprising:
   from 90 to 99.6% by weight of the hydrophilic phase, of at least one hydrophilic compound A and
   from 0.4 to 10% by weight of the hydrophilic phase, of at least one polymer P prepared by at least one polymerisation reaction:
      of at least one anionic monomer (a1) comprising at least one polymerisable olefinic unsaturation and at least one carboxylic acid group or one of its salts and
      of at least one C1-C8 ester (a2) of a compound derived from an acid chosen among acrylic acid, methacrylic acid, maleic acid, itaconic acid and crotonic acid,
the addition, under stirring, of a lipophilic phase comprising at least one lipophilic compound B, to form a dispersion of lipophilic phase particles in the continuous hydrophilic phase, then
the addition, under stirring, of at least one pigment C chosen among the lipophilic mineral pigments, the hydrophilic mineral pigments, the lipophilic organic pigments, the hydrophilic organic pigments and combinations thereof.

Preferably according to the invention, the tinted cosmetic composition is a foundation composition or a complexion-correcting composition or a complexion-unifying composition. BB (blemish balm) or CC (colour control) creams are examples of tinted cosmetic compositions. Very advantageously, the method according to the invention makes it possible to avoid the use of any surface-active compounds when preparing the emulsion. A surface-active compound works by reducing the surface tension of the particles of lipophilic compound. A molecule of surface-active compound comprises two distinct portions: a lipophilic portion and a hydrophilic portion. This is not the case for the polymer P, which alternates various monomer residues without forming separate lipophilic and hydrophilic portions. The polymer P used according to the invention is not a surface-active compound; it does not lead to micelle formation and does not have the behaviour of a surface-active compound.

Avoiding surface-active compounds when preparing the emulsion according to the invention provides many advantages. As many surface-active compounds are solid at room temperature or at temperatures of up to 50° C. or higher, their use would require a heating step at high temperature that the method according to the invention makes it possible to avoid.

Within the composition according to the invention, the amounts of ingredients can vary quite widely. Preferably, the method of preparation according to the invention enables the preparation of a composition that comprises:
from 25 to 94% by weight, of hydrophilic phase,
from 5 to 50% by weight, of lipophilic phase and
from 1 to 25% by dry weight of pigment C,
   relative to the weight of the composition.

More preferably, the method of preparation according to the invention enables the preparation of a composition that comprises:
from 45 to 75% by weight, of hydrophilic phase,
from 20 to 40% by weight, of lipophilic phase and
from 5 to 15% by dry weight of pigment C,
   relative to the weight of the composition.

Similarly, the ingredients of the hydrophilic phase can vary. Preferably for the method according to the invention the hydrophilic phase comprises from 0.4 to 9% by weight or from 0.4 to 8% by weight, preferably from 0.5 to 10% by weight or from 0.5 to 9% by weight or from 0.5 to 8% by weight or even from 0.6 to 10% by weight or from 0.6 to 9% by weight or from 0.6 to 8% by weight, of polymer P relative to the amount of hydrophilic phase.

The use of the polymer P makes it possible to effectively prepare the hydrophilic phase of the tinted cosmetic composition according to the invention. In particular, the use of this polymer P makes it possible to control the viscosity of the hydrophilic phase. The viscosity of the hydrophilic phase is thus generally higher than the viscosity of water and it is lower than a value at which the hydrophilic phase loses its workability. Preferably for the method according to the invention, the hydrophilic phase has a Brookfield viscosity, measured at 25° C. at a speed of 60 rpm, greater than 100 mPa·s or greater than 500 mPa·s. Also preferably for the method according to the invention, the hydrophilic phase has a Brookfield viscosity, measured at 25° C. at a speed of 60 rpm, less than 15,000 mPa·s or less than 10,000 mPa·s. More preferably for the method according to the invention, the hydrophilic phase has a Brookfield viscosity, measured at 25° C. at a speed of 60 rpm, ranging from 100 to 15,000 mPa·s or ranging from 100 to 10,000 mPa·s.

Essentially according to the invention, the hydrophilic continuous phase comprises at least one hydrophilic compound A and at least one polymer P prepared by at least one polymerisation reaction of monomers (a1) and (a2). The polymer P used according to the invention is known as such. It can be prepared by known methods, in particular by a radical polymerisation reaction, for example a polymerisation reaction in an emulsion, in a dispersion or in a solution. The polymerisation can be carried out, in a solvent, in the presence of at least one initiator compound. As examples of initiator compounds, at least one compound can be used chosen among the azoic initiator compounds (for example azobisisobutyronitrile), a peroxide compound, preferably hydrogen peroxide, benzoyl peroxide, benzoyl hydroperoxide and mixtures thereof. Alkaline metal persulphates can also be mentioned, particularly sodium persulphate and potassium persulphate, ammonium persulphate, partially water-soluble peroxides, particularly succinic peracid, t-butyl hydroperoxide, cumyl hydroperoxide, persulphates combined with a copper ion, a ferrous ion, a sulphite ion or a bisulphite ion and mixtures thereof.

Aside from the various monomers, the method for preparing the polymer P generally uses at least one chain transfer agent, preferably chosen among mercaptan compounds, in particular mercaptan compounds comprising at least 4 carbon atoms such as butyl mercaptan, n-octyl mercaptan, n-dodecyl mercaptan, tert-dodecyl mercaptan, iso-octyl 3-mercaptopropionate.

According to the invention, the radical-initiating or radical-generating compound can therefore be combined with at least one controlled radical polymerisation transfer agent, in particular a RAFT (reversible addition-fragmentation chain transfer) transfer agent. Preferably, the reaction is a radical polymerisation reaction in emulsion.

Preferably according to the invention, monomer (a1) is chosen among acrylic acid, methacrylic acid, maleic acid, itaconic acid, crotonic acid, an acrylic acid salt, a methacrylic acid salt, a maleic acid salt, an itaconic acid salt, a crotonic acid salt and combinations thereof. Much more preferentially, monomer (a1) is chosen among acrylic acid, an acrylic acid salt, methacrylic acid, a methacrylic acid salt and combinations thereof.

Preferably according to the invention, monomer (a2) is a C₁-C₆ ester or a C₁-C₄ ester. Also preferably according to the invention, monomer (a2) is a C₁-C₇ acrylic acid ester or a C₁-C₇ methacrylic acid ester, more preferentially chosen among methyl acrylate, ethyl acrylate, propyl acrylate, butyl acrylate, ethylhexyl acrylate, methyl methacrylate, ethyl methacrylate, propyl methacrylate, butyl methacrylate, ethylhexyl methacrylate and combinations thereof. More preferentially, monomer (a2) is chosen among ethyl acrylate, methyl acrylate, butyl acrylate, methyl methacrylate and combinations thereof.

Particularly preferably according to the invention, monomer (a1) is chosen among acrylic acid, methacrylic acid, maleic acid, itaconic acid, crotonic acid, an acrylic acid salt, a methacrylic acid salt, a maleic acid salt, an itaconic acid salt, a crotonic acid salt, and combinations thereof, much more preferentially acrylic acid or methacrylic acid; and monomer (a2) is a C₁-C₆ ester or a C₁-C₄ ester or is a C₁-C₇ acrylic acid ester or a C₁-C₇ methacrylic acid ester, preferably chosen among methyl acrylate, ethyl acrylate, propyl acrylate, butyl acrylate, ethylhexyl acrylate, methyl methacrylate, ethyl methacrylate, propyl methacrylate, butyl methacrylate, ethylhexyl methacrylate and combinations thereof, more preferentially ethyl acrylate, methyl acrylate, butyl acrylate, methyl methacrylate and combinations thereof.

More preferably according to the invention, monomer (a1) is chosen among acrylic acid, methacrylic acid and combinations thereof and monomer (a2) is an ester chosen among ethyl acrylate, methyl acrylate, butyl acrylate, methyl methacrylate and combinations thereof.

Also preferably, the polymerisation reaction uses:
from 20 to 75 mol % or from 25 to 70 mol %, preferably from 30 to 70 mol % or from 35 to 65 mol %, of monomer (a1) and
from 25 to 80 mol % or from 30 to 75 mol %, preferably from 30 to 70 mol % or from 35 to 65 mol %, of monomer (a2),
   relative to the total molar amount of monomers (a1) and (a2).

In addition to monomers (a1) and (a2), the polymer P can be prepared from other monomers. Thus, the polymer P can be prepared by a polymerisation reaction that also uses:
at least one compound (a3) chosen among 2-acrylamido-2-methylpropane sulphonic acid, ethoxymethacrylate sulphonic acid, sodium methallyl sulphonate, styrene sulphonate hydroxyethyl acrylate phosphate, hydroxypropyl acrylate phosphate, hydroxyethylhexyl acrylate phosphate, hydroxyethyl methacrylate phosphate, hydroxypropyl methacrylate phosphate, hydroxyethylhexyl methacrylate phosphate, their salts and combinations thereof or
at least one compound (a4) chosen among hydroxyethyl acrylate, hydroxypropyl acrylate, hydroxyethylhexyl acrylate, hydroxyethyl methacrylate, hydroxypropyl methacrylate, hydroxyethylhexyl methacrylate or
at least one cross-linking monomer (a5) or at least one monomer (a5) comprising at least two olefinic unsaturations or
at least one associative monomer (a6).

Also advantageously according to the invention, the polymerisation reaction can use less than 20 mol %, preferably from 0.2 to 20 mol %, particularly from 0.5 to 10 mol %, of monomer (a3) relative to the total molar amount of monomers.

Also advantageously, the polymerisation reaction can use less than 20 mol %, preferably from 0.2 to 20 mol %, particularly from 0.5 to 10 mol %, of monomer (a4) relative to the total molar amount of monomers.

According to the invention, monomer (a5) can be chosen among:
a compound of formula (I):

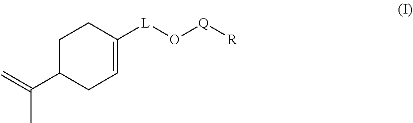

wherein:
L represents CH₂, CH₂ monoalkoxylate or CH₂ polyalkoxylate, preferably a CH₂ monoethoxylate group or a CH₂ polyethoxylate group,
Q represents a direct bond or C(O),
R represents —C(H)═CH₂, —C(CH₃)═CH₂, —C(H)═C(H)C(O)OH, —C(H)═C(H)CH₃, —C(═CH₂)CH₂C(O)OH, —CH₂C(═CH₂)C(O)OH, Q³OQ⁴OC(O)C(CH₃)═CH₂ or Q³OQ⁴OC(O)C(H)═CH₂,
Q³ represents a difunctional residue of an asymmetric diisocyanate compound, preferably chosen among tolyl-1,3-diisocyanate (TDI) and isophorone-diisocyanate (IPDI) and
Q⁴ represents CH₂, CH₂—CH₂, CH₂ monoalkoxylate, CH₂—CH₂ monoalkoxylate, CH₂ polyalkoxylate or CH₂—CH₂ polyalkoxylate and
a compound of formula (II):

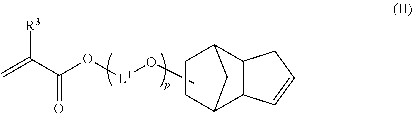

wherein:
R³ independently represents H or CH₃,
L¹ independently represents a straight or branched C₁-C₂₀-alkylene group, preferably an ethylene group or a propylene group and
p independently represents 0 or an integer ranging from 1 to 30, for example from 1 to 20, particularly from 1 to 15, in particular from 1 to 10.

Monomer (a5) can also be chosen among di(meth)acrylates such as polyalkylene glycol di(meth)acrylate, in particular polypropylene glycol di(meth)acrylate, ethylene glycol di(meth)acrylate, polyethylene glycol di(meth)acrylate, triethylene glycol di(meth)acrylate, 1,3-butylene glycol di(meth)acrylate, 1,6-butylene glycol di(meth)acrylate, 1,6-hexanediol di(meth)acrylate, neopentyl glycol di(meth)acrylate, 1,9-nonanediol di(meth)acrylate, but also 2,2′-bis(4-(acryloxy-propyloxyphenyl))propane, 2,2′-bis(4-(acryloxydiethoxy-phenyl))propane and zinc acrylate; tri(meth)acrylate compounds such as trimethylolpropane tri(meth)acrylate and ethoxylated trimethylolpropane tri(meth)acrylate, trimethylolethane tri(meth)acrylate, pentaerythritol tri(meth)acrylate and tetramethylolmethane tri(meth)acrylate; tetra(meth)acrylate compounds such as di-trimethylolpropane tetra(meth)acrylate, tetramethylolmethane tetra(meth)acrylate and pentaerythritol tetra(meth)acrylate; hexa(meth)acrylate compounds such as dipentaerythritol hexa(meth)acrylate; penta(meth)acrylate compounds such as dipentaerythritol penta(meth)acrylate; allyl compounds such as allyl (meth)acrylate, diallyl phthalate, diallyl itaconate, diallyl fumarate, diallyl maleate; polyallyl sucrose ethers with from 2 to 8 groups per molecule, pentaerythritol polyallyl ethers such as pentaerythritol diallyl ether, pentaerythritol triallyl ether and pentaerythritol tetraallyl ether; trimethylolpropane polyallyl ethers such as trimethylolpropane diallyl ether (TMPDAE) and trimethylolpropane triallyl ether. Other polyunsaturated compounds include divinyl glycol, divinyl benzene, divinylcyclohexyl and methylenebisacrylamide.

Monomer (a5) can also be prepared by an esterification reaction of a polyol with an unsaturated anhydride such as acrylic anhydride, methacrylic anhydride, maleic anhydride, or itaconic anhydride.

Monomer (a5) can also be chosen among the trifunctional cross-linking agents. This can be in particular trimethylolpropane tri(meth)acrylate (TMPTA) or ethoxylate trimethylolpropane tri(meth)acrylate (such as TMPTA 3EO).

Monomer (a5) can also be chosen among trimethylolpropane tri(meth)acrylate, ethoxylated trimethylolpropane tri(meth)acrylate, ethylene glycol di(meth)ethylacrylate, methylenebisacrylamide, diallyl phthalate, diallyl maleate, trimethylolpropane diallyl ether (TMPDAE) and mixtures thereof.

Monomer (a5) can also be a mixture of two separate monomers, such as EGDCPEA (ethylene glycol dicyclopentenyl ether acrylate) and TMPTA or EGDCPEA and TMPTA 3EO or even EGDCPEMA (ethylene glycol dicyclopentenyl ether methacrylate) and TMPTA or EGDCPEMA and TMPTA 3EO.

According to the invention, monomer (a5) is preferably chosen among a compound of formula (I), a compound of formula (II), trimethylolpropane trimethacrylate, trimethylolpropane triacrylate, ethoxylated trimethylolpropane trimethacrylate, ethoxylated trimethylolpropane triacrylate, ethylene glycol dimethacrylate, ethylene glycol diacrylate, methylenebisacrylamide, diallyl phthalate, diallyl maleate, trimethylolpropane diallyl ether (TMPDAE) and mixtures thereof.

Also advantageously, the polymerisation reaction can use less than 5 mol %, preferably from 0.01 to 4 mol %, particularly from 0.02 to 4 mol % or from 0.02 to 2 mol %, in particular from 0.02 to 1 mol %, of monomer (a5) relative to the total molar amount of monomers.

According to the invention, the associative monomer (a6) can be chosen among a compound of formula (III):

R¹—(EO)ₘ—(PO)ₙ—R² (III)

wherein:
m and n, identical or different, independently represent 0 or an integer or decimal less than 150, m or n is different from 0,
EO independently represents a CH₂CH₂O group,
PO independently represents a group chosen among CH(CH₃)CH₂O and CH₂CH(CH₃)O,
R¹ represents a group comprising at least one polymerisable olefinic unsaturation, preferably an acrylate group or a methacrylate group and
R² represents a straight or branched C₆-C₄₀-alkyl group, a phenyl group, a polyphenyl group, preferably a straight or branched C₁₀-C₃₀-alkyl group, more preferentially a straight or branched C₁₂-C₂₂-alkyl group, or a group comprising 2 to 5 phenyls or a tristyrylphenyl group or a pentastyrylcumylphenyl group.

Also advantageously according to the invention, the polymerisation reaction can use less than 20 mol %, preferably from 0.05 to 20 mol %, particularly from 0.1 to 10 mol %, of monomer (a6) relative to the total molar amount of monomers.

Preferably, the polymer P is prepared from the sole monomers (a1) to (a5), particularly from combinations of the sole monomers (a1)-(a2), (a1)-(a2)-(a3), (a1)-(a2)-(a4), (a1)-(a2)-(a5), (a1)-(a2)-(a3)-(a4), (a1)-(a2)-(a3)-(a5), (a1)-(a2)-(a4)-(a5), (a1)-(a2)-(a3)-(a4)-(a5), (a1)-(a2)-(a5)-(a6).

More preferably, the polymer P is prepared from the sole monomers (a1) and (a2) or from the sole monomers (a1), (a2) and (a5) or even from the sole monomers (a1), (a2), (a5) and (a6).

Advantageously according to the invention, the polymerisation reaction uses:
from 19.9 to 66 mol % or from 29.8 to 66 mol %, preferably from 29.8 to 53 mol % or from 29.8 to 49 mol %, of monomer (a1),
from 30 to 80 mol % or from 40 to 70 mol %, preferably from 45 to 70 mol % or from 50 to 70 mol %, of monomer (a2) and
from 0.01 to 4 mol % or from 0.02 to 4 mol %, preferably from 0.02 to 2 mol % or from 0.02 to 1 mol %, of monomer (a5),
   relative to the total molar amount of these three monomers.

Preferably according to the invention, the polymer P can be fully or partially neutralised, preferably by means of at least one compound chosen among NaOH, KOH, ammonium derivatives, ammonia, amine bases, for example triethanolamine, aminomethyl propanol, or 2-amino-2-methyl-propanol (AMP) and combinations thereof.

Also preferably according to the invention, the polymer P can be fully or partially coacervated, more preferentially:
by reducing the pH, for example by reducing the pH to a value of less than 6.5, in particular by means of an acid compound, in particular by means of least one organic or inorganic acid compound, in particular an acid compound chosen among phosphoric acid, citric acid, glucono-lactone, lactic acid, salicylic acid, glycolic acid, ascorbic acid, glutamic acid, hydrochloric acid, acetic acid, D-gluconic acid, sulphonic acid, methanesulphonic acid, benzimidazole sulphonic acid, tartaric acid, 4-aminobenzoic acid, benzoic acid, sorbic acid, phenylbenzimidazole sulphonic acid, benzylidene camphor sulphonic acid, terephthalylidene dicamphor sulphonic acid, kojic acid, hyaluronic acid or
by increasing the ionic strength, for example by adding at least one ionised compound or at least one salt, in particular NaCl, KCl, MgCl₂, CaCl₂, MgSO₄, CaSO₄, or by adding phenylbenzimidazole sulphonic acid (PBSA) or pyroglutamic acid sodium salt (NaPCA) or even by adding at least one ionised organic sun filter.

The method of preparation according to the invention is particularly simple and versatile. It thus makes it possible to prepare tinted cosmetic compositions whose composition can vary. It can be carried out under simple conditions and using standard equipment, in particular by means of commonly-used stirring or mixing apparatuses. Thus, for the method of preparation according to the invention, stirring is generally carried out at a minimum speed of 100 rpm or at a maximum speed of 3,000 rpm. Preferably, stirring is carried out by means of a device chosen among mixers equipped with spiral blades, mixers equipped with turbines (for example, deflocculating blades), ultrasonic mixers.

Particularly preferably according to the invention, the pH of the hydrophilic phase is modified, preferably the pH of the hydrophilic phase is modified to a value ranging from 6 to 8, in particular to a value ranging from 6 to 7 or from 6.5 to 7. According to the invention, the modification of the pH of the hydrophilic phase is carried out before the addition of the lipophilic phase. According to the invention, the pH of the hydrophilic phase can be modified by means of at least one compound chosen among NaOH, KOH, ammonium derivatives, ammonia, amine bases, for example triethanolamine, aminomethyl propanol or 2-amino-2-methyl-propanol (AMP) and combinations thereof, preferably by means of NaOH.

Preferably, the addition of the pigment C is performed at a temperature ranging from 0 to 50° C. or ranging from 5 to 35° C. or at room temperature.

Advantageously, the final pH of the tinted cosmetic composition according to the invention can be adjusted. Preferably, the pH is adjusted to a pH ranging from 4 to 13 or from 4 to 12 or from 4 to 11, more preferentially to a pH ranging from 5 to 13 or from 5 to 12 or from 5 to 11. Much more preferentially, the pH is adjusted to a pH ranging from 6 to 13 or from 6 to 12 or from 6 to 11.

The invention also relates to the tinted cosmetic composition obtained by the method of preparation according to the invention. The invention therefore also provides a tinted cosmetic composition in the form of an oil-in-water emulsion prepared by the method of preparation according to the invention. The tinted cosmetic composition according to the invention comprises the essential ingredients defined for this method of preparation.

In the tinted cosmetic composition according to the invention, the lipophilic phase is dispersed within the hydrophilic phase which comprises at least one hydrophilic compound A. Preferably according to the invention, the hydrophilic compound A is chosen among water alone or in a mixture with at least one compound chosen among the cosmetic moisturising compounds, glycerol, polyglycerols, glycols, propylene glycol, butylene glycol, sorbitol, sugar derivatives, for example xylitol, maltitol, urea, amino acids, lactic acid, pyroglutamic acid, hyaluronic acid, glycine and combinations thereof.

The tinted cosmetic composition according to the invention comprises at least one lipophilic compound B in the form of dispersed particles to form the lipophilic phase in the hydrophilic phase. Preferably, the lipophilic compound B is a cosmetic compound. Advantageously, the lipophilic compound B is liquid at room temperature. The lipophilic compound B can then be used without heating.

More preferably according to the invention, the lipophilic compound B is a synthetic substance or a natural substance, more preferentially the lipophilic compound B is chosen among the fatty acids, fatty alcohols, esters, butters, waxes (for example beeswaxes), oils, preferably an oil chosen among the mineral oils (for example paraffin oil, vaseline oil, mineral oils with a boiling point ranging from 300 to 400° C.), animal oils (for example squalenes, squalane, perhydrosqualene), vegetable oils (for example sweet almond oil, calophyllum oil, palm oil, apricot kernel oil, avocado oil, jojoba oil, olive oil, castor oil, cereal germ oils, the liquid fraction of shea butter), unsaponifiable compounds derived from natural oils, synthetic oils (for example hydrogenated polyisobutene, fatty acid esters such as purcellin oil, butyl myristate, isopropyl myristate, cetyl myristate, isopropyl palmitate, butyl stearate, hexadecyl stearate, isopropyl stearate, octyl stearate, isocetyl stearate, decyl oleate, hexyl laurate, propylene glycol dicaprylate, esters derived from lanolic acid such as diisopropyl lanolate, isocetyl lanolate, acetyl glycerides, octanoate alcohols, octanoate polyalcohols, decanoate alcohols, decanoate polyalcohols in particular octanoate glycols, octanoate glycerols, decanoate glycols, decanoate glycerols, ricinoleate alcohols, ricinoleate polyalcohols), terpenes, polyterpenes, phytosterol, silicone oils (for example cyclomethicones, low-molecular weight polydimethylsiloxanes or silicone oils, high-molecular weight polydimethylsiloxanes or silicone gums, polymethylsiloxanes, dimethiconols, phenyl polydimethylsiloxanes, low-molecular weight siloxanols, high-molecular weight siloxanols, trimethylsiloxysilicates), fluorinated oils (for example fluorinated perfluoroethers and fluorinated silicones); more preferably the lipophilic compound is chosen among the vegetable oils (for example, sweet almond oil, calophyllum oil, palm oil, apricot kernel oil, avocado oil, jojoba oil, olive oil, castor oil, cereal germ oils, the liquid fraction of shea butter), and the silicone oils (for example, cyclomethicones, low-molecular weight polydimethylsiloxanes or silicone oils, high-molecular weight polydimethylsiloxanes or silicone gums, polymethylsiloxanes, dimethiconols, phenyl polydimethylsiloxanes, low-molecular weight siloxanols, high-molecular weight siloxanols, trimethylsiloxysilicates).

Advantageously, the versatility of the method of preparation according to the invention makes it possible to use lipophilic compounds whose use in tinted cosmetic compositions is generally complicated or impossible. In such cases, the lipophilic compounds used are then finally chosen among the silicone oils. Advantageously, the method of preparation according to the invention therefore makes it possible to reduce the amount of or dispense with silicone oils in the tinted cosmetic composition prepared.

Preferably, the lipophilic compound B is then chosen among the vegetable oils, in particular chosen among sweet almond oil, calophyllum oil, palm oil, apricot kernel oil, avocado oil, jojoba oil, olive oil, castor oil, cereal germ oils, the liquid fraction of shea butter.

The lipophilic compound B can advantageously be chosen according to its sensory qualities or according to the pigment C.

Pigment C is an essential ingredient of the tinted cosmetic composition prepared according to the invention. More preferably according to the invention, the pigment C is chosen among the iron oxides, hydrophobically treated iron oxides, hydrophilically treated iron oxides, titanium dioxide, hydrophobically treated titanium dioxide, hydrophilically treated titanium dioxide, polysulphurized sodium aluminosilicate (ultramarine blue) and combinations thereof. The preferred examples of pigments C are chosen among iron oxides and iron hydroxides, in particular produced by synthesis and primarily composed of anhydrous iron oxides or hydrated iron oxides. The range of shades comprises yellow iron oxides, red iron oxides, brown iron oxides and black iron oxides. Anatase titanium dioxide, hydrated iron (III) oxide, hydrated ferric oxide, diiron trioxide, triiron tetraoxide are particularly effective as pigment C for the preparation of the tinted cosmetic composition according to the invention.

Particular pigment C combinations can be used. The preferred pigment C combinations are combinations of titanium dioxide and of iron oxides, particularly combinations chosen among:
titanium dioxides and iron oxides surface-treated with:
   alginate,
   silica,
   methicone,
   dimethicone,
titanium dioxides and iron oxides predispersed in:
   dimethicone,
   PEG,
   triethoxysilylethyl polydimethylsiloxyethyl dimethicone,
titanium dioxides and iron oxides predispersed in natural oils such as jojoba esters, polyglyveryl-6 polyricinoleate, jojoba oil (Simmondsia chinensis).

Particularly advantageously according to the invention, the pigment C can be present separately in the hydrophilic phase or in the lipophilic phase or can be present simultaneously in the hydrophilic phase and in the lipophilic phase. In general according to the invention, the pigment C is used directly. It can also be used in the form of a dispersion, particularly in the form of a dispersion in an oil. The preferred dispersion oils for the pigment C are chosen among the dimethicones, cyclosiloxanes, caprylic/capric triglycerides and jojoba vegetable oil.

Particular combinations of pigments C dispersed in non-D5 silicone oils, thus different from decamethylcyclopentasiloxanes (D5 silicone oils), can be chosen among:
titanium dioxide (for example, at 64% of solids content) dispersed in a dimethicone mixture, PEG/PPG-18/18 dimethicone, isopropyl titanium triisostearate, triethoxysilylethyl polydimethylsiloxyethyl dimethicone and tocopheryl acetate,
yellow iron oxides (for example, at 55% of solids content) dispersed in a dimethicone mixture, PEG/PPG-18/18 dimethicone, isopropyl titanium triisostearate, triethoxysilylethyl polydimethylsiloxyethyl dimethicone and tocopheryl acetate,
red iron oxides (for example, at 55% of solids content) dispersed in a dimethicone mixture, PEG/PPG-18/18 dimethicone, isopropyl titanium triisostearate, triethoxysilylethyl polydimethylsiloxyethyl dimethicone and tocopheryl acetate,
black iron oxides (for example, at 59% of solids content) dispersed in a dimethicone mixture, PEG/PPG-18/18 dimethicone, isopropyl titanium triisostearate, triethoxysilylethyl polydimethylsiloxyethyl dimethicone and tocopheryl acetate.

Advantageously, the method of preparation according to the invention makes it possible to obtain a tinted cosmetic composition whose viscosity can be controlled. Preferably according to the invention, the composition has a Brookfield viscosity, measured at 25° C. at a speed of 6 rpm, greater than 1,500 mPa·s. More preferably according to the invention, the composition has a Brookfield viscosity, measured at 25° C. at a speed of 6 rpm, ranging from 1,500 to 120,000 mPa·s.

Also advantageously, the pH of the tinted cosmetic composition prepared according to the invention can be controlled. Preferably according to the invention, the composition has a pH greater than 4, preferably greater than 5, more preferentially greater than 6 or 6.5. Also preferably according to the invention, the composition has a pH of less than 13, preferably less than 12, more preferentially less than 11. More preferably according to the invention, the composition has a pH ranging from 4 to 13 or from 4 to 12 or from 4 to 11, preferably ranging from 5 to 13 or from 5 to 12 or from 5 to 11, more preferentially ranging from 6 to 13 or from 6 to 12 or from 6 to 11, also more preferentially ranging from 6.5 to 13 or from 6.5 to 12 or from 6.5 to 11.

The tinted cosmetic composition according to the invention can also comprise at least one UV-stabilizing agent. Preferably, the UV-stabilizing agent can be chosen among:
titanium dioxide particles, zinc oxide particles, coated mineral particles, in particular coated with a hydrophobic compound, for example stearic acid, in particular titanium dioxide particles coated with a hydrophobic compound, for example stearic acid, and combinations thereof or
lipophilic UV-stabilizing agents, preferably chosen among avobenzone, ethylhexyltriazone, benzophenone-3, octocrylene, benzophenone-2, benzophenone-4, ethylhexylsalicylate, 4-methylbenzylidene camphor, octyl-N,N-dimethyl PABA, ethylhexyl dimethyl PABA, ethylhexyl methoxycinnamate, isoamyl methoxycinnamate, butyl methoxydibenzoylmethane, diethylamino hydroxybenzoyl hexyl benzoate, bis-ethylhexyloxyphenol methoxyphenyl triazine, drometrizole trisiloxane, methylene bis-benzotriazolyl tetramethylbutylphenol, diethylhexyl butamido triazone, ethylhexyl triazone, isoamyl p-methoxycinnamate, polysilicone-15, tris biphenyl triazine, homomenthyl salicylate, PEG-25 PABA, active lipophilic cosmetic molecules, active water-soluble cosmetic molecules, and combinations thereof.

The tinted cosmetic composition according to the invention can also comprise fillers (for example, calcium and aluminium borosilicates, mica, talc), microspheres (for example, nylon 12), hydrophilic neutralising agents, hydrophilic active ingredients (for example, hydrophilic UV filters), anti-oxidant agents (for example, tocopherol acetate, ascorbic acid), hydrating agents.

The invention also provides a method for controlling the stability of a tinted cosmetic composition, in the form of an oil-in-water emulsion prepared in the absence of surface-active compounds. The method for controlling the stability of a tinted cosmetic composition, in the form of an oil-in-water emulsion prepared in the absence of surface-active compounds, comprises:
a continuous hydrophilic phase comprising at least one hydrophilic compound A,
a lipophilic phase, comprising at least one lipophilic compound B, and dispersed in the form of particles in the continuous hydrophilic phase and
at least one pigment C chosen among the lipophilic mineral pigments, hydrophilic mineral pigments, hydrophilic organic pigments, lipophilic organic pigments and combinations thereof,
   by addition, during the preparation of the hydrophilic phase, of 0.4 to 10% by weight of the hydrophilic phase, of at least one polymer P prepared by at least one polymerisation reaction:
of at least one anionic monomer (a1) comprising at least one polymerisable olefinic unsaturation and at least one carboxylic acid group or one of its salts and
of at least one C₁-C₈ ester (a2) of a compound derived from an acid chosen among acrylic acid, methacrylic acid, maleic acid, itaconic acid and crotonic acid.

Due to its properties, the tinted cosmetic composition according to the invention is very easy to use, in particular as foundation, for correcting the complexion or for unifying the complexion. The invention thus provides a make-up method comprising the application to the skin of at least one tinted cosmetic composition according to the invention.

Particularly advantageously, the make-up method according to the invention enables excellent coverage of the surface of the skin by means of the tinted cosmetic composition of the invention. According to the invention, the covering power of the tinted cosmetic composition is determined by the index R=Y1/Y2, which is the luminance ratio measured on two parts of an unvarnished contrast card comprising a black part and a white part (Leneta FORM N2A-2 Opacity) covered with a layer of composition 200 μm thick:
Y1: luminance of the tinted composition applied to the black part of the contrast card and
Y2: luminance of the tinted composition applied to the white part of the contrast card.

Preferably, the make-up method according to the invention has a covering power whose R index is greater than 0.75 or greater than 0.85 or greater than 0.90.

The make-up method according to the invention also allows for excellent retention of the tinted cosmetic composition on the skin. In particular, the make-up method according to the invention has good resistance to washing. According to the invention, the resistance to washing by bi-permuted water or by saline water (NaCl at 0.9 g/l) is determined by measuring the spreading diameter D of a drop of water deposited on a layer of tinted cosmetic composition 200 μm thick. Preferably, the make-up method according to the invention achieves a spreading diameter D of less than 20 mm, preferably less than 15 mm or less than 13 mm. The make-up method according to the invention can achieve a spreading diameter D of less than 13 mm by using a tinted cosmetic composition comprising a silicone oil. The make-up method according to the invention can also achieve a spreading diameter D of less than 10 mm by using a tinted cosmetic composition that is free of silicone oil.

According to the invention, the particular, advantageous or preferred characteristics of the method for preparing the tinted cosmetic composition according to the invention define tinted cosmetic compositions according to the invention as well as make-up methods which are also particular, advantageous or preferred.

The following examples illustrate the various aspects of the invention.

### EXAMPLES

### Preparation of Polymers P1 and P2 According to the Invention and of a Comparative Polymer CP1

### Polymer P1

The following compounds are used:
MAA: methacrylic acid (monomer a1),
EA: ethyl acrylate (monomer a2),
2-[(3a,4,5,6,7,7a-hexahydro-4,7-methano-1H-inden-6-yl)oxy]ethyl acrylate Fancryl FA 512 AS (F) (monomer a5),
Huntsmann Empicol LXVN: sodium lauryl sulphate (SLS),
BASF Texapon NSO: sodium laureth sulphate in 28% solution or sodium lauryl ether sulphate (SLES) in 28% solution,
sodium persulphate (NH₄)₂S₂O₈,
n-dodecyl mercaptan,
C₁₆ isomethacrylate ethoxylated 25 times (from a Guerbet alcohol) (monomer (a6)). In a stirred 1L reactor heated using an oil bath, mixture 1 is prepared by introducing deionised water and a sodium lauryl sulphate (SLS) solution or an aqueous solution containing 28% by mass of sodium lauryl ether sulphate (SLES).

A mixture 2 is prepared in a beaker, called a monomer premixture, comprising in deionised water:
monomer (a1), methacrylic acid (MAA),
monomer (a2), ethyl acrylate (EA),
monomer (a5),
a 28% solution of sodium lauryl ether sulphate (SLES) or of sodium lauryl sulphate (SLS),
optionally an associative monomer (a6) of formula (III), C₁₆EO25-isomethacrylate,
optionally a transfer agent, n-dodecyl mercaptan.

This premixture is stirred to form a monomer mixture.

An initiator solution is prepared comprising ammonium persulphate and deionised water and optionally sodium metabisulphite. All reagents and amounts used are listed in Table 1. For polymer (P1) used according to the invention, the reactor is heated to 85° C.±1° C., the initiator solution is injected and the monomer premixture is injected in parallel over 2 hours and 30 minutes. This mixture is cooked for 60 min at 88° C.±1° C. and then cooled to room temperature.

For polymer (P2) used according to the invention, the reactor is heated to 85° C. ±1° C., all of the initiator solution is injected as soon as the reactor has come to the set temperature and the monomer premixture is injected in parallel over 2 hours and 30 minutes. This mixture is cooked for 60 min at 88° C.±1° C. and then cooled to room temperature.

The polymers according to the invention were prepared under these conditions by varying the monomer compositions of the monomer premixtures. The copolymer compositions obtained are shown in Table 1.

TABLE 1

| | | |
|---|---|---|
| Amount (g) | P1 | P2 |

| | | | |
|---|---|---|---|
| Mixture 1 | deionised water | 400 | 574.9 |
| | SLS | 2.60 | 0 |
| | SLES | 0 | 10.46 |
| Premixture | deionised water | 173.70 | 22.98 |
| | SLS | 1.81 | 0 |
| | MAA (a1) | 105.69 | 88.84 |
| | EA (a2) | 191.28 | 176.36 |
| | monomer (a5) | 2.75 | 0.88 |
| | monomer (a6) | 0 | 22.35 |
| | n-dodecyl mercaptan | 0 | 0.18 |
| Initiator | deionised water | 54.32 | 62.85 |
| | ammonium persulphate | 0.58 | 0.41 |
| Composition | monomer (a1) (mol %) | 38.98 | 36.68 |
| | monomer (a2) (mol %) | 60.67 | 62.63 |
| | monomer (a5) (mol %) | 0.35 | 0.13 |
| | monomer (a6) (mol %) | 0 | 0.56 |

| | | |
|---|---|---|
| Final solids content (% by weight) | 30 | 29 |

### Preparation of Tinted Cosmetic Composition Formulations F1 to F10 According to the Invention and Comparative CF1 and CF2

### Ingredients

Hydrophilic Phase
Hydrophilic compounds:
   A1: bi-permuted water
   A2: glycerol
   A3: propylene glycol
   A4: butylene glycol
Polymers P1, P2, CP1

Lipophilic Phase
Silicone oils:
   B1: dimethicone (Xiameter PMX-200 Silicone Fluid 5cS)
   B2: cyclopentasiloxane and cyclohexasiloxane (Xiameter 345 Fluid)
Natural oils:
   B3: jojoba vegetable oil
   B4: coconut vegetable oil (hydrogenated and hydrolysed caprylic/capric triglyceride derivative) (DUB MCT)

Pigments
Untreated pigments:
   C1: anatase titanium dioxide (Pigment White 6 titanium dioxide CI77891)
   C2: iron III oxide hydrate (ferric oxide hydrate) (Pigment Yellow 42 iron oxides CI 77492)
   C3: diiron trioxide (Pigment Black 11 iron oxides CI77491)
   C4: triiron tetraoxide (Pigment Red 101 iron oxides CI77499)
Pigments hydrophilically surface-treated with alginate:
   C5: alginate-treated titanium dioxide and aluminium hydroxide (Miyoshi MiyoAQUA White TSR)
   C6: alginate-treated iron oxide, red (Miyoshi MiyoAQUA Red)
   C7: alginate-treated iron oxide, yellow (Miyoshi MiyoAQUA Yellow)
   C8: alginate-treated iron oxide, black (Miyoshi MiyoAQUA Black NH)
Pigments hydrophilically surface-treated with silica:
   C9: titanium dioxide and aluminium hydroxide surface-treated with silica (Kobo SIH-2 TiO2 R250)
   C10: iron oxide surface-treated with silica (Kobo CI77492 SIH-2 Yellow No. 602P)
   C11: iron oxide surface-treated with silica (Kobo CI77491 SIH-2 Red No. 211P)
   C12: iron oxide surface-treated with silica (Kobo CI77499 SIH-2 Black No. 710P)
Pigments hydrophobically treated with methicone:
   C13: titanium dioxide treated with methicone (Kobo CI77891 BTD-MS2)
   C14: iron oxide treated with methicone (Kobo CI77492 BXYO-MS2)
   C15: iron oxide treated with methicone (Kobo CI77491 BXRO-MS2)
   C16: iron oxide treated with methicone (Kobo CI77499 BXBO-MS2)
Pigments hydrophobically treated with dimethicone:
   C17: titanium dioxide treated with dimethicone (Kobo BTD-DS4)
   C18: iron oxide treated with dimethicone (Kobo BYO-DS4 CI77492)
   C19: iron oxide treated with dimethicone (Kobo BRO-DS3 CI77491)
   C20: iron oxide treated with dimethicone (Kobo BBO-DS3 CI77499)
Pigments hydrophobically treated with natural oils:
   C21: titanium dioxide treated with silica and natural Simmondsia chinensis oil, jojoba oil esters and polyglyveryl-6 polyricinoleate (Kobo CI77891 JOH65UJE)
   C22: iron oxide treated with silica and natural Simmondsia chinensis oil, jojoba oil esters and polyglyveryl-6 polyricinoleate (Kobo CI77492 JOH45YJE)
   C23: iron oxide treated with silica and natural Simmondsia chinensis oil, jojoba oil esters and polyglyveryl-6 polyricinoleate (Koba CI77491 JOH55RJE)
   C24: iron oxide treated with silica and natural Simmondsia chinensis oil, jojoba oil esters and polyglyveryl-6 polyricinoleate (Koba CI77499 JOH55BJE)

### Additional Ingredients

Sodium hydroxide in 20% aqueous solution

Preservative: phenoxyethanol

Formulations F1 and F2 of Cosmetic Composition Tinted with Untreated Pigments According to the Invention

Different mixtures of ingredients are prepared beforehand using a Raynerie motor (VMI) equipped with a spiral mixing blade or a dispersion blade (deflocculating blade). The ingredients and amounts used are shown in Table 2.

Mixture I: At room temperature, under stirring using a mixing blade, the hydrophilic phase ingredients are mixed in the order shown in the table, and the pH is adjusted to 6.8-6.9 using sodium hydroxide (20% in water) in an amount sufficient (QSP) to reach this pH range. Then, the speed is gradually increased between 600 and 800 rpm for 5 minutes.

Mixture II: mixture Ito which the ingredients of the lipophilic phase are added, one by one and in the order of the table, while maintaining stirring.

Mixture III: mixture II to which a preservative is added while increasing stirring to 1,200 rpm.

Mixture IV: mixture III in which the pigments (pre-dispersed separately beforehand by mixing under stirring with a deflocculating blade at 2,500 rpm for 4 minutes in an oil) are dispersed, using a deflocculating blade, by increasing the stirring to 3,000 rpm for 10 minutes.

TABLE 2

Formulations

| Mixtures | Ingredients | F1 | F2 |
|---|---|---|---|
| I | A1 | QSP 100% | QSP 100% |
| | P2 | 5.0 | — |
| | P1 | — | 4.0 |
| | Sodium hydroxide | QSP pH 6.8 | QSP pH 6.8 |
| | A2 | 10.0 | 10.0 |
| | A3 | 3.0 | 3.0 |
| | A4 | 1.0 | 1.0 |
| II | B1 | 9.0 | 9.0 |
| III | Phenoxyethanol | 0.5 | 0.5 |
| IV | B2 | 19.0 | 19.0 |
| | C1 | 6.09 | 6.09 |
| | C2 | 2.98 | 2.98 |
| | C3 | 0.51 | 0.51 |
| | C4 | 0.62 | 0.62 |

Formulations F3 and F4 of Cosmetic Composition Tinted with Hydrophilic Pigments on the Surface Using Alginate According to the Invention

Mixture I and mixture II are prepared in a manner similar to the preparation of formulation F1, under stirring at 1,200 rpm, mixture III and mixture IV with pre-dispersion of the pigments under stirring at 2,500 rpm for 5 minutes and mixing under stirring at 3,000 rpm for 5 minutes. The ingredients and amounts used are shown in Table 3.

TABLE 3

Formulations

| Mixtures | Ingredients | F3 | F4 |
|---|---|---|---|
| I | Al | QSP 100% | QSP 100% |
| | P2 | — | 5.0 |
| | P1 | 3.0 | |
| | Sodium hydroxide | QSP pH 6.8 | QSP pH 6.8 |
| II | B1 | 9.0 | 9.0 |
| | B2 | 19.0 | 19.0 |
| III | Phenoxyethanol | 0.5 | 0.5 |
| IV | A2 | 10.0 | 10.0 |
| | A3 | 3.0 | 3.0 |
| | A4 | 1.0 | 1.0 |
| | C5 | 5.00 | 5.00 |
| | C6 | 3.97 | 3.97 |
| | C7 | 0.61 | 0.61 |
| | C8 | 0.42 | 0.42 |

Formulations F5 and F6 of Cosmetic Composition Tinted with Hydrophilic Pigments on the Surface Using Silica According to the Invention

Mixture I, mixture II, mixture III and mixture IV are prepared in a manner similar to the preparation of formulation F3. The ingredients and amounts used are shown in Table 4.

TABLE 4

Formulations

| Mixtures | Ingredients | F5 | F6 |
|---|---|---|---|
| I | A1 | QSP 100% | QSP 100% |
| | P2 | 5.0 | — |
| | PI | — | 4.0 |
| | Sodium hydroxide | QSP pH 6.8 | QSP pH 6.8 |
| II | B1 | 28.0 | 28.0 |
| III | Phenoxyethanol | 0.5 | 0.5 |
| IV | A2 | 10.0 | 10.0 |
| | A3 | 3.0 | 3.0 |
| | A4 | 1.0 | 1.0 |
| | C9 | 5.0 | 5.0 |
| | C10 | 3.97 | 3.97 |
| | C11 | 0.61 | 0.61 |
| | C12 | 0.42 | 0.42 |

Formulations F7 to F10 of Cosmetic Compositions Tinted with Hydrophobically Treated Pigments Using Dimethicone or Methicone According to the Invention

Mixture I is prepared in a manner similar to the preparation of formulation F1, under stirring at 500 rpm, mixture II, mixture III and mixture IV with pre-dispersion of the pigments under stirring at 2,500 rpm for 5 minutes and lastly mixture V. The ingredients and amounts used are shown in Table 5.

TABLE 5

| | | |
|---|---|---|
| Mix- | Ingre- | Formulations |

| tures | dients | F7 | F8 | F9 | F10 |
|---|---|---|---|---|---|
| I | Al | 30.0 | 30.0 | 30.0 | 30.0 |
| | P2 | — | 5.0 | — | 5.0 |
| | Pi | 4.0 | — | 4.0 | — |
| | Sodium | QSP | QSP | QSP | QSP |
| | hydroxide | pH 6.8 | pH 6.8 | pH 6.8 | pH 6.8 |
| II | B1 | 15.0 | 15.0 | 15 | 15 |
| III | Phenoxy- | 0.5 | 0.5 | 0.5 | 0.5 |
| | ethanol | | | | |
| IV | B1 | 13.0 | 13.0 | 13.0 | 13.0 |
| | C13 | 5.00 | 5.00 | — | — |
| | C14 | 3.97 | 3.97 | — | — |
| | C15 | 0.61 | 0.61 | — | — |
| | C16 | 0.42 | 0.42 | — | — |
| | C17 | — | — | 5.00 | 5.00 |
| | C18 | — | — | 3.97 | 3.97 |
| | C19 | — | — | 0.61 | 0.61 |
| | C20 | — | — | 0.42 | 0.42 |
| V | A2 | 10.0 | 10.0 | 10.0 | 10.0 |
| | A3 | 3.0 | 3.0 | 3.0 | 3.0 |
| | A4 | 1.0 | 1.0 | 1.0 | 1.0 |
| | Al | QSP | QSP | QSP | QSP |
| | | 100% | 100% | 100% | 100% |

Formulation F11 of a Cosmetic Composition Tinted with Hydrophobically Treated Pigments Using Natural Oils According to the Invention

Mixture I is prepared in a manner similar to the preparation of formulation F1, under stirring at 500 rpm, mixture II, mixture III and mixture IV with pre-dispersion of the pigments under stirring at 3,000 rpm for 3 minutes. The ingredients and amounts used are shown in Table 6.

TABLE 6

| Mixtures | Ingredients | Formulation F11 |
|---|---|---|
| I | Al | 40 |
| | P1 | 0 |
| | P2 | 4 |
| | Sodium hydroxide | 0.84 |
| II | B4 | 10 |
| III | Phenoxyethanol | 0.6 |
| IV | C21 | 17.45 |
| | C22 | 1.73 |
| | C23 | 1.18 |
| | C24 | 0.2 |
| | B3 | 5 |
| | Al | 19.03 |

### Evaluation of the Properties of Formulations F1 to F11 and CF1

The properties of the formulations prepared using the tinted cosmetic compositions according to the invention are compared with the properties of a comparative cosmetic formulation CF1. Comparative formulation CF1 is a commercial tinted cream (Miss Dem BB Cream 5 in 1) in the form of an oil-in-water emulsion prepared from a silicon oil. It consists of the following ingredients: water, ethylhexyl methoxycinnamate, glycerol, phenyl trimethicone, steareth-2, benzophenone-3, caprylic/capric triglyceride, cetearyl alcohol, dimethicone, isononyl isononanoate, steareth-21, magnesium aluminium silicate, hydrogenated polyisobutene, benzyl alcohol, phenoxyethanol, ethylhexylglycerol, sodium dehydroacetate, xanthan gum, fragrance, dehydroacetic acid, disodium EDTA, propylene/ethylene/styrene copolymer, Rhodiola rosea root extract, sodium hyaluronate, butylene/ethylene/styrene copolymer, carnosine, coral powder, BHT, alanyl-glutamine, pigment CI 77891, pigment CI 77492, pigment CI 77491, pigment CI 77499.

### Evaluation of the Stability of the Formulations

The various formulations are stored in a transparent container for one month at a temperature of 50° C. and at room temperature. The stability of the formulations is visually determined by the presence of any phase separation. No dephasing is observed for formulations F1 to F11 according to the invention when stored at room temperature or at 50° C.

### Evaluation of the Retention of the Formulations on a Substrate

The water repellency of a formulation allows for good affinity with a hydrophobic substrate such as the skin. The formulations are assessed for their resistance to rain or sweat. The water repellent properties of the formulations are evaluated with deionised water for rain and with saline water (0.9 g/L NaCl aqueous solution) for sweat.

Using an applicator (Elcometer, Model 3540), the formulation is applied to a glass plate to form a 0.05 mm thick film which is allowed to dry for approximately 24 hours to obtain a touch-dry strand.

A drop of water is deposited on the formulation film from a height of 1 cm using a 3 mL pipette. Then the diameter (mm) of the circle of the film into which the water has diffused is measured and this value is taken to determine the composition's water repellency. As the value decreases, the composition will be increasingly water-repellent and will have better retention after application to the skin. Diameter d1 (mm) is measured for deionised water and diameter d2 (mm) is measured for saline water. The results are shown in Table 7.

TABLE 7

| | Formulations | | | | | | | | | |
|---|---|---|---|---|---|---|---|---|---|---|
| Properties | F1 | F2 | F3 | F4 | F5 | F6 | F7 | F10 | F11 | CF1 |
| d1 | 9.5 | 9 | 12 | 7 | 10 | 8 | 8 | 10 | 8 | 14 |
| d2 | 8 | 8 | 10 | 8 | 9 | 7 | 7 | 9 | 7 | 13 |

The formulations comprising the tinted cosmetic compositions according to the invention have deionised water and salt water repellent properties that are far superior to those of the comparative composition. The retention of these formulations on the skin will therefore be much better.

### Evaluation of the Covering Power of the Formulations

The covering power of the formulations is determined by a method derived from standard NF-T 30-075 for measuring the hiding power of paints and varnishes. The covering power is determined by the index R=Y1/Y2, which is the luminance ratio measured on two parts of an unvarnished contrast card comprising a black part and a white part (Leneta FORM N2A-2 Opacity) covered by a 200 μm thick layer of formulation:
Y1: luminance of the formulation applied to the black part and
Y2: luminance of the formulation applied to the white part.

The covering power or coverage is optimal for an R-ratio value close to 1. These measurements are carried out by means of a spectrophotometer (Byk Sphere Gloss) with the following settings: observer 10°, illuminant D65, colour reference Y,x,z., geometry d/8. Using an applicator (Elcometer Model 3540), the formulations are applied in a 200 μm thick layer to an unvarnished, black and white chart (Leneta Model FORM N2A-2—Opacity) and dried for approximately 24 hours prior to the luminance measurements. The results are shown in Table 8.

TABLE 8

Formulations

| Property | F1 | F4 | F5 |
|---|---|---|---|
| R | 1.2 | 1.02 | 1.04 |

The covering power of the formulations comprising the tinted cosmetic compositions according to the invention by means of pigments is excellent both for the compositions comprising a silicone oil and for the compositions not comprising a silicone oil but an oil of natural origin.

1. A method for preparing a tinted cosmetic composition as an oil-in-water emulsion prepared in the absence of surface-active compounds, the method comprising:
preparing a continuous hydrophilic phase comprising (i) a hydrophilic compound A in a range of
from 90 to 99.6 wt. % of a hydrophilic phase of the oil-in-water emulsion, and (ii) a polymer P, in a range of
from 0.4 to 10 wt. % of the hydrophilic phase, the polymer P comprising, in polymerized form,
an anionic monomer (a1) comprising a polymerizable olefinic unsaturation and a carboxylic acid group, optionally in salt form, and
a C₁-C₈ ester (a2) of acrylic acid, methacrylic acid, maleic acid, itaconic acid and/or crotonic acid;
adding, under stirring, a lipophilic phase comprising a lipophilic compound B, to form a dispersion of lipophilic phase particles in the continuous hydrophilic phase; then
adding, under stirring, of a pigment C comprising a lipophilic mineral pigment, hydrophilic mineral pigment, lipophilic organic pigment, and/or hydrophilic organic pigment.

2. The method of claim 1, wherein the tinted cosmetic composition comprises, relative to total composition weight:
the hydrophilic phase in a range of from 25 to 94 wt. %;
the lipophilic phase in a range of from 5 to 50 wt. %; and
the pigment C in a range of from 1 to 25 wt. % dry.

3. The method of claim 1, wherein the monomer (a1) is acrylic acid, methacrylic acid, maleic acid, itaconic acid, crotonic acid, an acrylic acid salt, a methacrylic acid salt, a maleic acid salt, an itaconic acid salt, a crotonic acid salt, or a combination thereof, or
wherein the monomer (a2) is a C₁-C₆ ester, or
wherein the monomer (a1) is acrylic acid, methacrylic acid, maleic acid, itaconic acid, crotonic acid, an acrylic acid salt, a methacrylic acid salt, a maleic acid salt, an itaconic acid salt, a crotonic acid salt, or a combination thereof, and and the monomer (a2) is a C₁-C₆ ester, or
wherein the polymer P is made by polymerizing, relative to total moles of the monomers (a1) and (a2):
the monomer (a1) in a range of from 20 to 75 mol. %; and
the monomer (a2) in a range of from 25 to 80 mol. % or from 30 to 75 mol % preferably from 30 to 70 mol % or from 35 to 65 mol %.

4. The method of claim 1 wherein the hydrophilic phase comprises the polymer P in a range of from 0.4 to 9 wt. % relative to total hydrophilic phase weight, or
wherein the hydrophilic phase has a Brookfield viscosity, measured at 25° C. at a speed of 60 rpm, greater than 100 mPa·s.

5. The method of claim 1 4, wherein the hydrophilic compound A is water, optionally combined with glycerol, polyglycerols, glycols, propylene glycol, butylene glycol, sorbitol, sugar derivatives, for example, xylitol, maltitol, urea, amino acids, lactic acid, pyroglutamic acid, hyaluronic acid, glycine, or a combination thereof.

6. The method of claim 1,
wherein the lipophilic compound B is liquid at room temperature, or
wherein the lipophilic compound B is a synthetic substance or a natural substance.

7. The method of claim 1, wherein the pigment C is iron oxide, hydrophobically treated iron oxide, hydrophilically treated iron oxide, titanium dioxide, hydrophobically treated titanium dioxide, hydrophilically treated titanium dioxide, polysulfurized sodium aluminosilicate, or a combination thereof.

8. The method of claim 1, wherein the pigment C is present separately in the hydrophilic phase or in the lipophilic phase or is present simultaneously in the hydrophilic phase and in the lipophilic phase.

9. The method of claim 1, wherein the tinted cosmetic composition has:
a Brookfield viscosity, measured at 25° C. at a speed of 6 rpm, greater than 1,500 mPa·s; or
a pH greater than 4.

10. The method of claim 1, wherein the polymer P is prepared by a polymerization reaction further comprising:
a compound (a3) which is 2-acrylamido-2-methylpropane sulfonic acid, ethoxymethacrylate sulfonic acid, sodium methallyl sulfonate, styrene sulfonate, hydroxyethyl acrylate phosphate, hydroxypropyl acrylate phosphate, hydroxyethylhexyl acrylate phosphate, hydroxyethyl methacrylate phosphate, hydroxypropyl methacrylate phosphate, hydroxyethylhexyl methacrylate phosphate, or a combination thereof, optionally as salt(s); or
a compound (a4) which is hydroxyethyl acrylate, hydroxypropyl acrylate, hydroxyethylhexyl acrylate, hydroxyethyl methacrylate, hydroxypropyl methacrylate, or hydroxyethylhexyl methacrylate; or
a cross-linked monomer (a5) or monomer (a5) comprising a first and a second olefinic unsaturation; or
an associative monomer (a6); or
the polymer P is at least partially neutralized; or
the polymer P is at least partially coacervated.

11. The method of claim 1, wherein the stirring in the adding of the lipophilic phase and/or the pigment C is carried out at a minimum speed of 100 rpm,
wherein the pH of the hydrophilic phase is modified, or
wherein the adding of the pigment C is carried out at a temperature in a range of from 0° C. to 50° C., or
wherein a final pH of the tinted cosmetic composition is adjusted.

12. A tinted cosmetic composition in the form of an oil-in-water emulsion, prepared by the method of claim 1.

13. A method for controlling the stability of a tinted cosmetic composition, as an oil-in-water emulsion prepared in the absence of surface-active compounds, the method comprising:
adding, during preparation of a hydrophilic phase, a polymer P in a range of from 0.4 to 10 wt. % of the hydrophilic phase,
wherein the polymer P comprises, in polymerized form:
an anionic monomer (a1) comprising a polymerizable olefinic unsaturation and a carboxylic acid group, optionally in salt form; and
a C₁-C₈ ester (a2) of acrylic acid, methacrylic acid, maleic acid, itaconic acid, and/or crotonic acid,
wherein the tinted cosmetic composition comprises:
a continuous hydrophilic phase comprising a hydrophilic compound A as the hydrophilic phas;
a lipophilic phase, comprising a lipophilic compound B, dispersed as particles in the continuous hydrophilic phase; and
a pigment C which is a lipophilic mineral, hydrophilic mineral, hydrophilic organic, lipophilic organic pigment, or combination thereof.

14. A make-up method, comprising:
applying the tinted cosmetic composition of claim 12 to skin.

15. The make-up of claim 14, wherein the covering power index R is greater than 0.75,
wherein the covering power index R is Y1/Y2, which is the luminance ratio measured on two parts of an unvarnished contrast card comprising a black part and a white part covered with a layer of the tinted cosmetic composition 200 μm thick,
wherein Y1 is luminance of the tinted cosmetic composition applied to the black part of the contrast card and Y2 is luminance of the tinted cosmetic composition applied to the white part of the contrast card.

16. The make-up method of claim 14, wherein a spreading diameter D of a drop of water deposited on a layer of the tinted cosmetic composition 200 μm thick is less than 20 mm.

17. The method of claim 1, wherein the monomer (a1) is acrylic acid, an acrylic acid salt, methacrylic acid, a methacrylic acid salt, or a combination thereof.

18. The method of claim 1, wherein the monomer (a2) is a C₁-C₄ ester.

19. The method of claim 1, wherein the monomer (a2) is a C₁-C₇ acrylic acid ester or a C₁-C₇ methacrylic acid ester.
